(19) United States
(12) Patent Application Publication
(10) Pub. No.: US 2015/0237327 A1
(43) Pub. Date: Aug. 20, 2015

(54) Process for creating a three dimensional x-ray image using a single x-ray emitter

(71) Applicant: 3-D XRay Technologies, L.L.C., Lee's Summit, MO (US)

(72) Inventors: Wade Anthony Rustici, Lee's Summit, MO (US); Mark Wade Grubb, Kansas City, MO (US); James Benjamin Dye, Raymore, MO (US)

(73) Assignee: 3-D XRay Technologies, L.L.C., Lee's Summit, MO (US)

(21) Appl. No.: 14/545,402

(22) Filed: Apr. 30, 2015

(51) Int. Cl. H04N 13/02; G06T 7/00; H04N 5/32; A61B 6/04; A61B 6/00

(52) U.S. Cl. CPC H04N 13/0207; A61B 6/0421; A61B 6/5211; A61B 6/466; H04N 5/32; G06T 7/0024; G06T 2207/10012; G06T 2207/10116; G06T 2207/30196

(57) ABSTRACT

A subject, such as a patient, is restrained in a desired position for X-raying a body part of interest and the X-ray emitter is activated to create a beam of X-rays that pass through the subject and into a digital X-ray detector to create an image. Then the X-ray detector is refreshed for another exposure and the X-ray emitter is moved from its first position to a second position and a second X-ray is taken. Computer software manipulates the two images by aligning them, interlacing them and warping one image into the space of the other image. The resulting image can be immediately displayed on a computer or the like or stored as a computer file, such as a JPEG file for later viewing. The images can be viewed as three dimensional images when the viewer wears three dimensional viewing eyeglasses.

### CROSS-REFERENCE TO RELATED APPLICATIONS

Not Applicable.

### STATEMENT REGARDING FEDERALLY SPONSORED RESEARCH OR DEVELOPMENT

Not applicable.

### SEQUENCE LISTING

Not applicable

### BACKGROUND OF THE INVENTION

The present invention is related to an apparatus and process for creating a three dimensional X-ray image using a single X-ray emitter by taking an image on an X-ray imaging medium with the X-ray emitter in a first position and then moving the X-ray emitter to a second position and taking another exposure on the same X-ray imaging medium. The X-ray subject, generally a part of a person, is held stationary during this process.

### DESCRIPTION OF THE RELATED ART INCLUDING INFORMATION DISCLOSED UNDER 37 C.F.R. 1.97 and 1.98

The utility of X-ray images can be substantially enhanced if they can be made to be three dimensional, or to appear to the viewer to be three dimensional. Substantial resources have been devoted to making three dimensional X-ray images, as reflected in numerous patents. It is important that the patient not move during the X-ray exposure, which is typically fairly easy to accomplish for one X-ray exposure. Two separate exposures are made over the surface of the entire X-ray receiver, which must be a digital X-ray receiver, or X-ray plate, in the present invention, to produce images that can be manipulated by computer software to create a computer file that can be displayed and viewed as a three dimensional image. If two separate spaced apart X-ray emitters are used, then patient or subject movement is typically not a significant problem because the exposure time is quite short. A subject can be either a human or animal or any other object that one might want to X-ray. Each exposure covers the entire surface of the X-ray receiver, but two different digital images are captured, one label L for left and the other labeled R for right, denoting the relative positions of the X-ray emitter when the images are captured.

Many medical offices, however, have X-ray equipment that has only a single X-ray emitter because a system with more than one X-ray emitter is generally perceived as unnecessary and as too expensive. Using X-ray equipment having a single X-ray emitter to produce three dimensional images requires that the X-ray emitter be moved between exposures, that is, a first image is made with the X-ray emitter in a first position and then the X-ray emitter is moved to a second position, generally by sliding it along conventional rails that are designed to permit adjustment of the position of the X-ray emitter, and a second exposure is made on the same X-ray medium. In this case, subject movement is a particular problem that compromises the quality of the resulting three dimensional images. In some cases, the resulting three dimensional image is blurred simply because the subject has moved laterally a little bit between exposures. In other cases, the entire view is compromised because the angle of the subject to the X-ray emitter have changed significantly. In either case, the two images cannot be completely aligned by computer software or other means, resulting in three dimensional images that are blurred and are harder to use in making a diagnosis and planning a course of treatment.

It has further been found that the only effective way to prevent misalignment of the two X-ray images due to subject movement during the time it takes to move the single X-ray emitter from a first position to a second position is to restrain the subject, locking the subject into the positioned to X-ray a particular part of the subject from a particular angle. It is especially difficult for a subject to hold still for even a few seconds if the subject is in pain, increasing the importance of a restraint system. It typically takes about five to seven seconds to move the X-ray emitter from a first position to a second position. If the subject moves a few inches or rotates more than about 3°-5°, the resulting three dimensional image is blurred enough that a diagnosis is harder to make and more problematic. X-rays are often taken with subjects contorted into unnatural positions, and often in several different positions and any effective restraint system must be able to restrain a subject in odd or difficult positions.

An example of a system having two separate X-ray emitters is found in U.S. Pat. No. 7,809,102 B2, issued to Brada et al. on Oct. 5, 2010, discloses a Method and Apparatus for Positioning a Subject in a CT Scanner comprising an MRI machine having a pair of spaced X-ray emitters separated by both aimed at a single detector array 208, which produces digital images, where they are received after passing through a subject on the support platform. The two sets of image data are combined and manipulated in an image processor to form an apparent three-dimensional image, which can be generated in any of a number of known fashions, including, for example displaying the two images on a single computer monitor in such a way that one image is visible only to the left eye and the other image is visible only to the right eye.

Another example of this type of system is found in U.S. Pat. No. 7,693,256 B2, issued to Brahme et al. on Apr. 6, 2010, discloses a Phase-Contrast X-ray Machine, comprising a pair of separate spaced apart X-ray sources 90, 100 projecting X-rays through a patient 110 and onto a single X-ray detector 40. The data from the X-ray detector is manipulated to produce a three-dimensional display.

In another approach, two separate images may be made on a single X-ray receiver and then the images are combined later. For example, U.S. Pat. No. 7,558,368 B2 issued to Klingenbeck-Regn on Jul. 7, 2009, discloses a Method for Creation of Stereo Image Pairs of an Object Under Examination with an X-ray System and X-ray System comprising using a single source of X-rays to make two exposures on a single X-ray receiver by tilting a body that carries both the X-ray source and the X-ray receiver in fixed spatial relationship to one another, relative to the patient. The resulting images are manipulated in a known manner that is not specified to produce stereoscopic images. This system is too large for most small offices and cannot be made smaller, as it requires a bed that the patient lies on, which also limits the types of images that can be made by it.

In many medical facilities, however, and especially in small practices, it is cost prohibitive to use an X-ray system that has two separate X-ray emitters. In these cases, producing three dimensional images that are clear is quite difficult due to patient movement between exposures, which is inevitable since it takes a significant amount of time to move the X-ray emitter to the desired second position. An example of a system for producing three dimensional images using a single X-ray emitter is found in U.S. Pat. No. 5,233,639 B1, issued to Marks on Aug. 30 1977, discloses a Stereoscopic Fluoroscopy Apparatus and Method of Producing Stereoscopic X-ray Images. Marks '639 discloses taking a first X-ray on a photographic plate behind the patient, shifting the X-ray apparatus laterally and taking a second exposure on the same photographic plate, then viewing the X-ray image with stereoscopic viewing equipment. This reference discloses moving a single source of X-rays, i.e., an X-ray tube, to take multiple images on a single X-ray receiver. The resulting image pairs are viewed as three-dimensional image through a stereoscopic viewing device.

An example of a system using a single X-ray emitter to make multiple exposures onto a single X-ray receiver is found in U.S. Pat. No. 6,317,481 B1 issued to Berestov on Nov. 13, 2001, which discloses Stereo X-ray Image Processing comprising an X-ray tube and X-ray film connected by a rod that holds the X-ray tube 110 and X-ray film in fixed relative spatial relationship so that they can be pivoted about an intermediate point, thereby rotating around the patient and making multiple images from different points onto the same X-ray medium for generating stereoscopic images. A computer rotates the images into the same planes, recalculates each point in the image and removes keystone distortion by locating matching points in the images and correcting the images and forming them into a single stereoscopic image. The optical axis of what would be the camera if one camera lens in one position could make a stereoscopic image is placed in the center of middle of the stereo image. This system is too large for most small offices and cannot easily be made smaller. Further, it will be difficult to maintain the X-ray emitter and the X-ray receiver in the same positions relative to one another. Inevitably the patient will move between images, blurring the resulting image.

Another example of using only one X-ray emitter to make two exposures on a single X-ray receiver is found in U.S. Pat. No. 6,256,372 B1, issued to Aufrichtig et al. on Jul. 3, 2001, which discloses an Apparatus and Methods for Stereo Radiography comprising an X-ray emitter mounted on a trolley for projecting X-rays onto an X-ray detector 14. The X-ray emitter 12 is moved from a first position to a second position along the trolley 20 and exposures taken at different points to generate left and right images. The X-ray emitter is rapidly moved to the second position. The two images are processed in a computer to create a stereoscopic image that can be viewed by users using eyeglasses that masks one image from view for each eye. A major disadvantage of this system is that the patient will inevitably move while the X-ray emitter is being moved, resulting in blurred images, which are always less useful than clear images would be.

To address the problem of patient movement in other types of systems, patient restraint systems have been developed as shown, for example, in U.S. Pat. No. 4,045,678 B1 issued to Rickard on Aug. 30, 1977, which discloses a Medical Restraint comprising boards and perpendicular section boards with attached straps closed with hook and loop type fasteners. This reference is a very large system, which is basically a bed with two straps that lie across the patient's body. This system is too large for many small offices and cannot be made smaller and, further, is limited to positions in which the patient is basically lying down, making it unsuitable for making images of a person's head or other body parts.

Therefore there is a need for a three dimensional X-ray system using a single X-ray emitter that produces X-ray images that are not blurred from patient movement between exposures. There is a need for such a system that produces high quality resolution three dimensional images using currently commonly available digital X-ray imaging equipment found in most medical offices. There is a need for such a system that readily permits taking one or more X-rays of different portions of a patient's body and for insuring that the patient does not move between exposures.

### BRIEF SUMMARY OF THE INVENTION

Accordingly, it is a primary object of the present invention to provide a process for creating three dimensional X-ray images that are not blurred.

It is a further object of the present invention to provide a process for creating three dimensional X-ray images utilizing an X-ray system having only one X-ray emitter.

It is another object of the present invention to provide a process for creating three dimensional X-ray images that includes preventing the subject from moving between X-ray exposures on the same X-ray medium.

Two separate images are produced on the same image media, preferably an electronic digital exposure plate that produces black and white images from X-rays passing through a person, with bones producing shadows, in the conventional manner, by taking one exposure and then moving the X-ray source a little bit, usually to one side from the original position and making another exposure. This is reminiscent of old stereotypes, but those used dual exposures from two cameras, i.e., light receivers, spaced a fixed distance apart. In the case of the present invention, the source of illumination, i.e., X-rays, is moved and two exposures are made onto a single fixed-in-place image media.

These two images are manipulated by computer software that permits full rotation of the images, tilting the images and so forth. This is accomplished by picking a number points of greatest contrast from the surrounding areas and using those points as locator points about which the other image elements are manipulated. The spatial relationship between the two images in maintained by keeping the dark locator points in each of the two images in the same relationship to one another during any manipulation.

The present invention also includes a mechanical apparatus that restrains the patient in a fixed position during the two exposures. If the patient were to move, the two images would not be properly aligned for three dimensional presentation.

The images are viewed through polarized three dimensional viewing glasses as are used in modern three dimensional movies. The left image is displayed only on the odd numbered horizontal lines of a polarized computer display panel and the right image is displayed only on the even numbered horizontal lines of the computer display. The polarized glasses allow the viewer's left eye to see the left image only, while the right eye sees only the right image. The viewer's brain then attempts to resolve these two different images into a single image, which it cannot, resulting in a three dimensional image. Alternate methods for viewing stereoscopic images in three dimensions exist and could be used. Examples of alternate methods includes active shutter glasses, or a head mounted display with a separate display for each eye and lenses used primarily to relax eye focus.

Presenting the X-ray image in three dimensions allows the viewer to see features that are not visible in two-dimensional X-ray images.

Other objects and advantages of the present invention will become apparent from the following description taken in connection with the accompanying drawings, wherein is set forth by way of illustration and example, the preferred embodiment of the present invention and the best mode currently known to the inventors for carrying out their invention.

### BRIEF DESCRIPTION OF THE SEVERAL VIEWS OF THE DRAWING

FIG. 1 is perspective view of the equipment and setting for implementing a process for creating a three dimensional X-ray image using a single X-ray emitter according to the present invention.

FIG. 2 is a top view of the equipment and setting for implementing a process for creating a three dimensional X-ray image using a single X-ray emitter of FIG. 1.

FIG. 3 is a side view of an interior portion of a pivoting plate for use with the process for creating a three dimensional X-ray image using a single X-ray emitter of FIG. 1.

FIG. 4 is an activity diagram of the software for use with the process for creating a three dimensional X-ray image using a single X-ray emitter of FIG. 1.

FIG. 5 is a use case diagram of the software for implementing the process for creating a three dimensional X-ray image using a single X-ray emitter of FIG. 1.

### DETAILED DESCRIPTION OF THE INVENTION

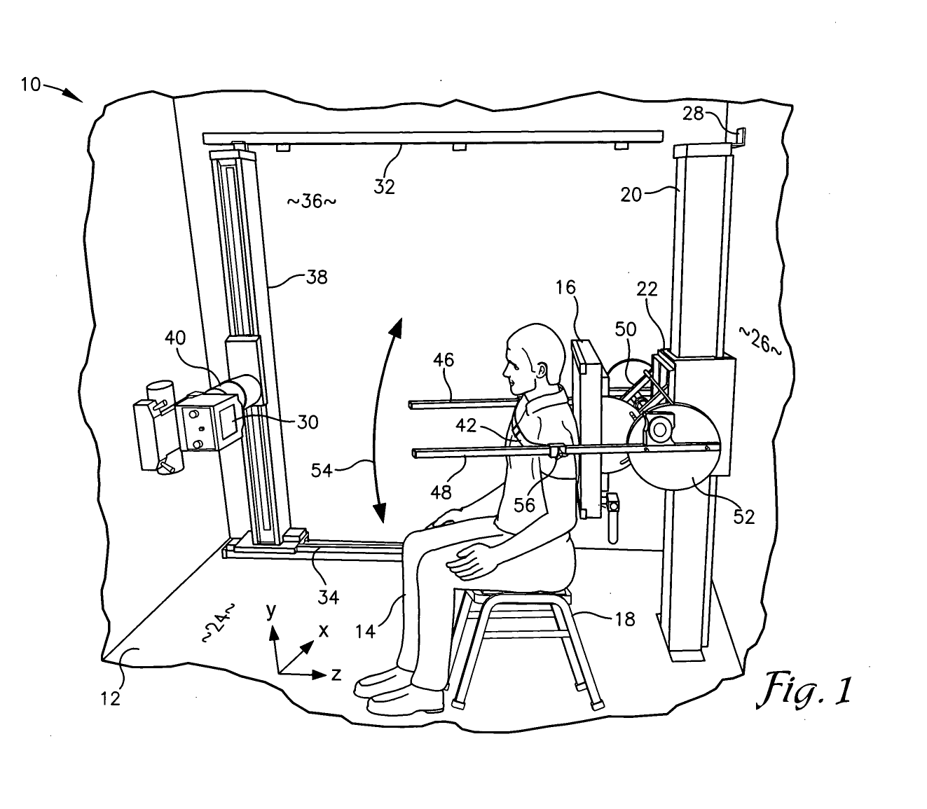

Referring to FIG. 1, the equipment and setting for implementing a process for creating a three dimensional X-ray image using a single x-ray emitter 3D X-ray process 10 according to the present invention is set up in a room 12 with a subject 14, in this case a person, pressed against a digital X-ray receiver, or detector (X-ray plate) 16. In this case, the subject 14 is seated on a chair 18, but other suitable means for supporting the subject 14, such as a stool, table or the like may also be used or the subject may be standing. The subject 14 should be as close to, and preferably pressed against, the digital X-ray detector 16 in order to minimize any magnification or distortion of the X-ray images, particularly any keystone effect. The position of the X-ray receiver 16 can be adjusted up or down along the supporting column 20 that it is connected to through the adjustment sleeve 22. The bottom end of the supporting column 20 is attached to the floor 24 and its top end is attached to the vertical rear wall 26 at the connector bracket 28. The position of the X-ray emitter 30 can be adjusted by moving it along a pair of spaced parallel horizontal tracks, that is the upper track 32 and the lower track 34. The upper track 32 is fastened to the right side wall 36 of the room 12 (with left and right in FIG. 1 defined from the position of the subject 14) and the lower track 34 is fastened to the floor 24. A vertical column 38 supports the X-ray emitter 30, which can be moved horizontally back and forth along the projecting horizontal adjustment arm 40. Thus, the position of the X-ray emitter 30 can be adjusted to essentially any desired position in three dimensions, allowing the X-ray emitter 30 to be positioned as needed to project X-rays through the subject 14 and into the X-ray plate 16. If desired, the X-ray emitter 30 can also be tilted. In general however, it is desirable to place the X-ray emitter 30 near the subject 14 with its face parallel to the X-ray plate 16 to minimize distortion.

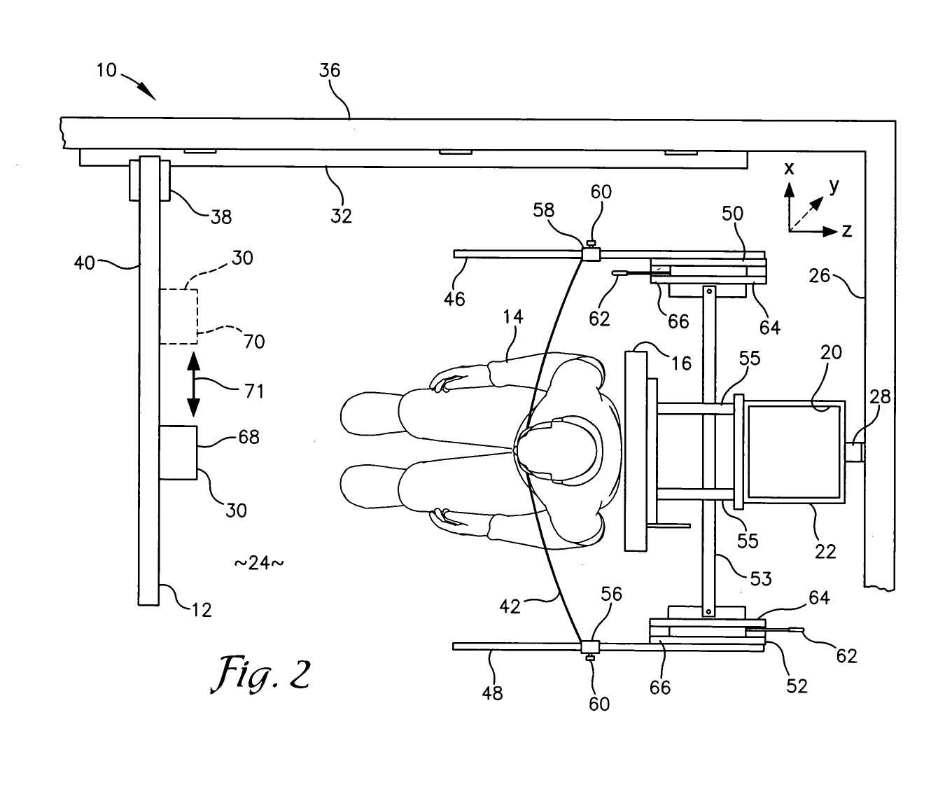

Still referring to FIG. 1, the subject 14 is restrained, or locked down, by the restraining strap 42. The restraining system 44 includes a right arm 46 and a left arm 48. The right arm 46 is fastened to a right pivot plate 50 and the left arm 48 is fastened to a left pivot plate 52. Each arm 46, 48 can be pivoted about their respective pivot plates 50, 52 independently of one another along the path of the vertically oriented double headed arrow 54. A left sliding collar 56 allows the restraining strap 42 to be moved to any desired position along the left arm 48 and then locked by a set screw. An identical arrangement allows the right end of the restraining strap 42 to be moved to any desired position along the length of the right arm 46. If desired, more than one restraining strap 42 can be attached to the arms 46, 48 to provide additional restraint. The pivot plates 50, 52 permit the angle of the respective arms 46, 48 to be pivoted in 15° increments through all 180° of rotation, but larger or smaller increments could be used. The pivot plates 50, 52 are fastened to a square tube mounting bar 53 (FIG. 2) that is horizontal and that is fastened to a pair of spaced parallel arms 55 (FIG. 2) connecting the X-ray plate 16 to the adjustment sleeve 22.

In using the 3D X-ray process 10, the medical practitioner first positions the subject 14 in the position that the medical practitioner has determined will best present the area of the subject 14 that needs to be diagnosed. Then each pivot or swivel arm 46, 48 is independently positioned at an angle that will provide the best traverse of the area of interest by the restraining strap 42 or straps and then adjusts the positions of the two sliding adjustment collars 56, 58 independently to provide the greatest amount of contact with the area of interest of the subject 14. Then the thumb setscrews 60 on the adjustment collars 56, 58 are tightened one at a time, so that, prior to tightening the whichever is the second thumb set screw 60 to be tightened, the restraining strap 42, which is threaded through the adjustment collars 56, 58, can be pulled tight before the thumb screw 60 is tightened, pinching the restraining strap 42 between the outer surface of an arm 46, 48 and the corresponding sliding adjustment collar 56, 58. Other tensioning means can be used, such as a winch disposed anywhere along the restraining strap 42 that does not contact the subject 14. Due to the ability to adjust the angle of either arm 46, 48 at any angle in 15° increments, or finer if desired, and the distance of the ends of the restraining strap 42 from the subject 14, any part of a subject 14, such as a patient, can be restrained and locked down against the X-ray detector 16 in any position determined to be desirable to the medical practitioner in making a diagnosis, or in allowing a chiropractor or physician to diagnosis later, insuring that both X-ray exposures are taken without the subject's 14 moving at all, providing superior X-ray images. Prior to tightening the restraining strap 42, the subject 14 is pressed against the X-ray detector 16.

Referring to FIG. 2, the right sliding adjustment collar 58 on the right arm 46 includes the thumb set screw 60, as does the left sliding adjustment collar 56, both of which allow the adjustment collars 56, 58 to be tightened at any desired point along either arm 46, 48 independently on one another as needed. The right pivot plate 50 and the left pivot plate 52 are identical in construction, with orientation of the adjustment lever 62 being different on the left side than on the right side only due to the distribution of adjustment apertures along only 180° of the circumference of the pivot plates 50, 52. Each pivot plate 50, 52 includes an inner plate 64 and an outer plate 66 with the adjustment lever 62 disposed between them.

Still referring to FIG. 2, the X-ray emitter 30 is first placed into the first position 68 and an X-ray image is taken by actuating the X-ray emitter 30. Then the X-ray detector 16 is readied for taking another image and the X-ray technician manually moves the X-ray emitter 30 into a second position 70 and a second X-ray image is taken by actuating the X-ray emitter a second time. Although the X-ray technician will move the X-ray emitter 30 quickly, it still takes five to eight seconds in practice. Clearly, the subject 14 would be likely to move during this period but for the restraining system 44, which would result the two different X-ray images being taken at different angles, spoiling the three dimensional image assembled later. The process of moving the X-ray emitter from a first position 68 to a second position 70 can be automated by, for example, forming a toothed rack along the horizontal arm 40 or providing suitable grooves on the horizontal arm 40 and groove-fitting idler or drive wheels on the X-ray emitter 30 and using one or more stepper motors or the like to drive the X-ray emitter 30 from one position to another, typically controlled by a computer program controlled by an operator who would enter the starting position for the first position and a second position for a desired second position. Automating the process of moving the X-ray emitter 30 between the first and second positions and in selecting the positions would be faster and more precise using the automated system but other factors tend to favor the use of the manually operated process described here.

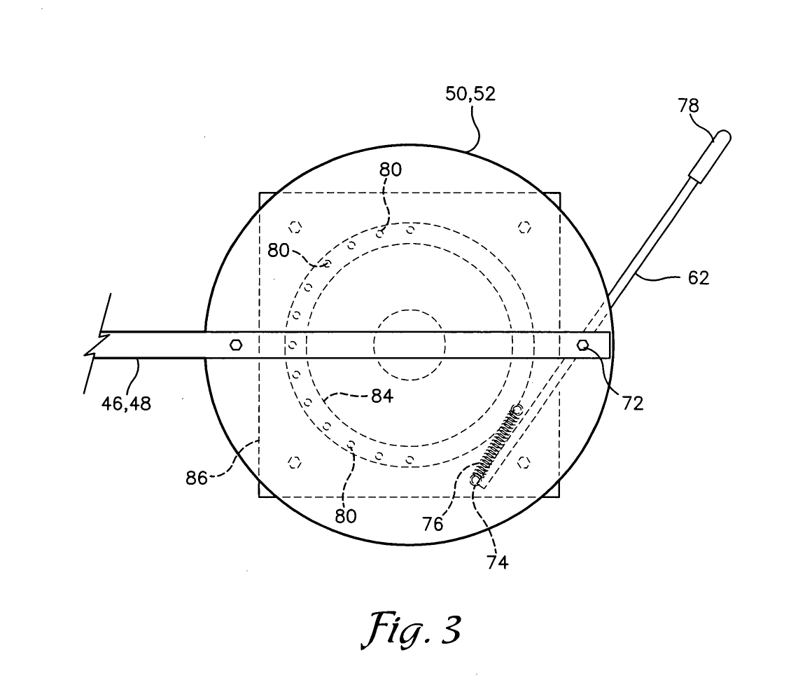

Referring to FIG. 3, each pivot plate 50, 52 includes the inner plate 64 to which the adjustment lever 62 pivots over the bolt 72 and has its distal end 78 fastened to a bracket 74 along with one end of a coil spring 76. Pushing the proximal end 78 of the adjustment lever 62 counterclockwise as shown causes a lug to disengage from a notch, allowing the pivot plate or swivel plate, to rotate. The operator can control how far the pivot plate 50, 52 pivots. A number of adjustment apertures 80 are distributed about the perimeter of a circular member 84 inside the square base 86 to which the outer circular disks are fastened. The design of the pivot or swivel plate 50, 52 is very common and well known and is commonly used in chairs, bar stools and the like. There are a number of conventional ways to make a swivel or pivot plate 50, 52, including, for example, a simple pin inserted into aligned holes in a pair of close concentric plates.

A variety of techniques for producing images that appear to the viewer to be three dimensional are well known. One method is disclosed in U.S. Pat. No. 6,256,372 B1, which is hereby incorporated by reference.

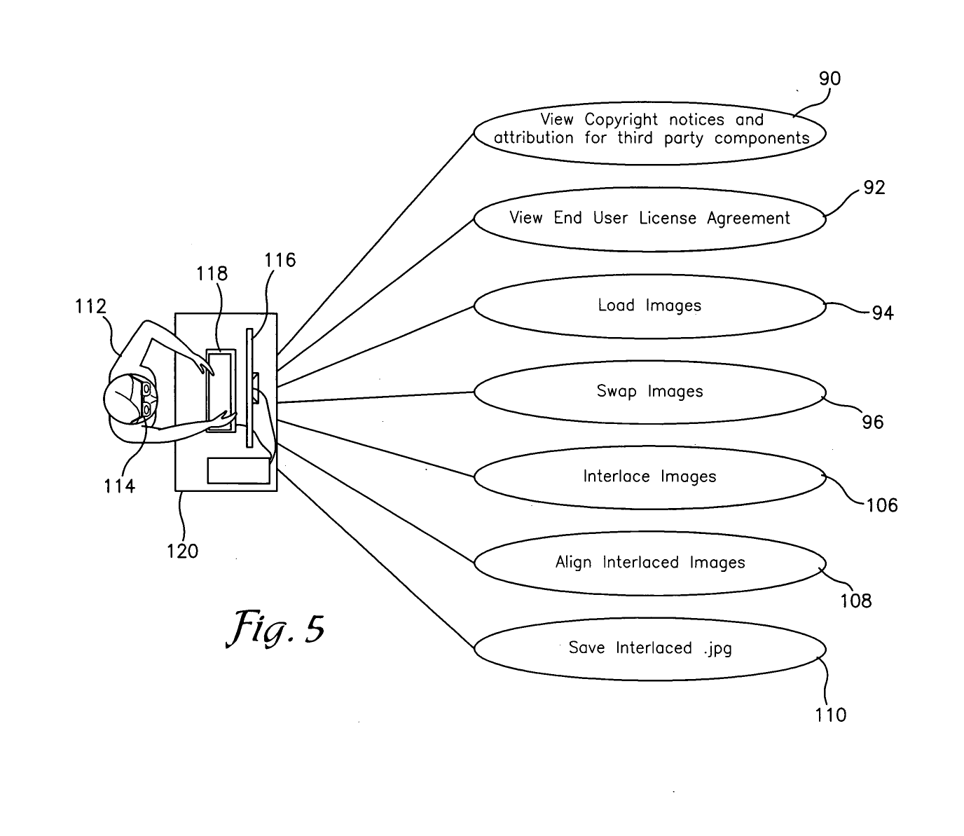

In orienting various images and things in space, the convention adopted here is to orient everything from the point of view of a person or user 112 (FIG. 5) viewing a computer monitor 116. Looking at the monitor 116, the X-axis is horizontal along the bottom edge of the monitor 116 and the Y-axis is vertical along the left hand side of the monitor 116. The Z-axis, used to denote depth, runs in and out of the monitor 116 and is perpendicular to both the X-axis and the Y-axis. This definition of XYZ space is carried over to FIGS. 1, 2, where it is represented in the XYZ icons, which indicated that the depth or Z-space is the same relative to the digital X-ray detector 116 as it is to the computer monitor 116.

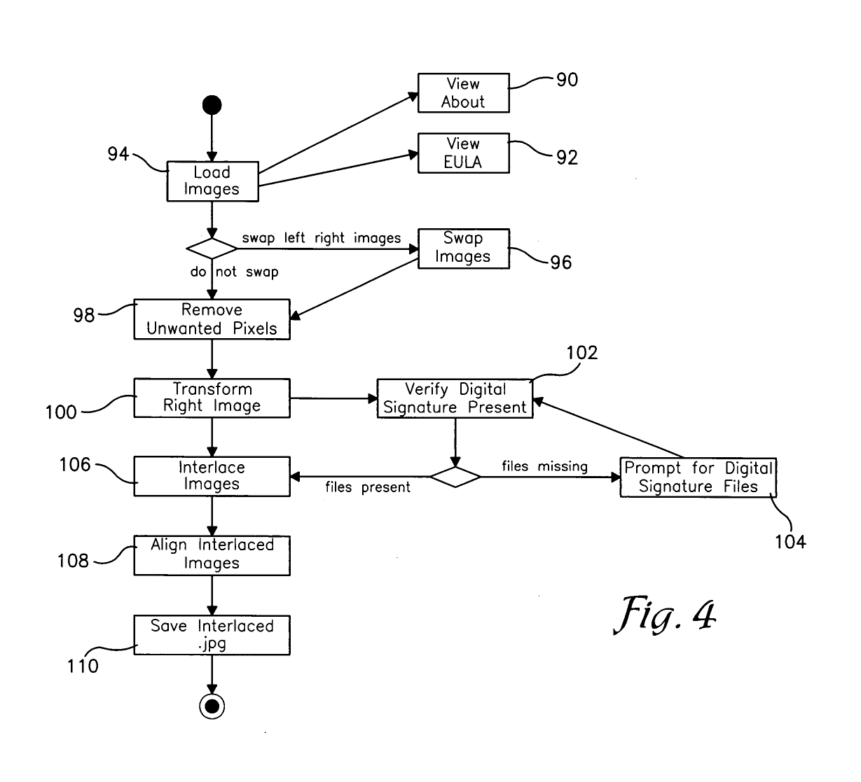

Referring to FIG. 4, the technique developed for the 3D X-ray process 10 includes the computer software that the user operates to create and view source images in three dimensional space and to create a computer file that can be saved in any computer medium, stored, transmitted and displayed for a chiropractor, physician other medical FIG professional in diagnosing conditions that cannot be diagnosed without visualizing systems or structures inside the body. The 3D X-ray process 10 is particularly adapted for use with human or animal subjects. A subject can be either a human or animal or any other object that one might want to X-ray. The software accepts two separate X-rays, a pair created from a single subject 14 with a shift in the Z-axis. This pair of X-ray images is aligned such that the only shift in coordinates is in the Z-axis, that is, from a first position 68 to a second position 70. The X-ray image loaded into the right pane is transformed to the X-axis and Y-axis coordinate space of the X-ray image loaded into the a left pane of the computer monitor 116. This process requires the identification of keypoints in each image. Keypoints, spelled as a single word, is a term of art in imaging. The keypoints are then used to find descriptors in each image. Complimentary pixels in images are matched using the descriptors from each image. The matches are filtered to remove poor matches. Additional filtering of the resultant matches removes additional false positives. Remaining good matches are used to transform the right image to the X-axis and Y-axis coordinate space of the left image. Prior to finding keypoints and immediately after loading source images, the left and right tags, typically in a corner of each image, are zeroed or replaced with black pixels. This removes the left and right markers and other pixels that are not part of the image of the subject 18. This significantly improves the resulting transformation. The resulting stereo pair is needed for viewing images in three dimensional space. The resulting image may be viewed as a three dimensional image or saved as a JPEG file or the like. The static saved image cannot be edited. Any adjustment or modification of the image required using the original source images, which would be manipulated by a user 112 using the software again.

Still referring to FIG. 4, the view about module 90 provides copyright notices and attribution for thirty party components. The view end user license agreement module 92 allows the user to view the end user license agreement, which must be agreed to before the user can proceed. If the user declines to accept the end user license agreement, a system exit is performed and program execution is terminated.

When the end user license agreement is accepted, the load images module 94 is invoked and the left and right digital X-ray images are loaded into a split pane by the file chooser. Typically the left image refers to the image produced when the X-Ray emitter 30 is shifted towards the left from the perspective of a person who is facing the X-ray detector 16, that is, into a second position 70 (FIG. 2). As shown by the double headed arrow 71, the X-ray emitter 30 can be moved along the horizontal adjustment arm 40. Similarly the right image refers to the image produced when the X-Ray emitter 30 is shifted towards the right relative to the X-ray detector 16 when facing the X-ray detector 16, that is, into the first position 68 (FIG. 2). Alternatively, the opposite terminology could be used in relationship to the X-ray detector 16 to define the left and right images, here the former definition is used. When the end user license agreement is accepted, the load images module 94 is invoked and the left and right digital X-ray images are loaded into a split pane by the file chooser.

A decision is made whether or not to swap the left and right digital X-ray images based on the viewer's needs. If yes, then the swap images module 96 is invoked and the left and right digital X-ray stereo images are swapped in the split pane, which supports inverting the positions of the right and left digital images. Typically the image will be viewed in parallel, meaning the left eye sees the left image and the right eye sees the right image. When swapped, this is referred to as cross-eyed or cross and the left eye sees the right image, and the right eye sees the left image. When viewed as cross-eyed, or cross, the three dimensional image is reversed, with features normally appearing to be at the back of the image appearing at the front, and features normally appearing to be at the front appearing to be in the back. If yes, then the left and right digital X-ray stereo images are swapped in position in the split pane, which supports inverting the positions of the right and left digital images. This swap can be useful to a medical practitioner who needs to view a particular structure in three dimensions in order to make a sound diagnosis. Requiring the user, or medical practitioner, to invoke the swap images module or decline to use it ensures that the user or medical practitioner knows for certain that the image that is being viewed was inverted from front to back.

Whether or not the user chooses to swap the positions of the two images, the remove unwanted pixels module 98 is invoked next. Whenever X-ray images are taken of a subject 14, a marker is placed in the image to denote the orientation of the subject. For example, if the subject is facing the X-ray emitter 30, a marker indicating ‘left’ could be placed on the left hand side of the subject in the image as this would sufficiently define the orientation of the subject during exposure. Alternatively, for that same image, a ‘right’ marker could have been placed on the right side of the subject to satisfactorily define the orientation of the subject 14. (Likewise, if the subject were facing away from the X-ray emitter 30, ‘left’ or ‘right’ markers could be appropriately placed in the image to define the orientation of the subject 14.) This is a requirement in all X-ray imaging to ensure the doctor or other medical practitioner is treating the appropriate side of the subject 14. Whenever stereoscopic pairs of images are taken of a subject 14, a marker is also required in each image to denote which image represents the ‘left’ image of the pair and which represents the ‘right’ image of the pair. The right or left labeling is typically made with a capital letter R for “right” and a capital letter L for “left” during exposure, but other markers could be used to define the stereo shift. Both markers, subject 14 orientation and stereo shift, must be utilized in creating a stereoscopic pair of X-ray images. These markers or labels, which must be unique markers so that the medical practitioner can distinguish the point of view of the X-ray image when viewing them separately, which may be desirable sometimes, must be ignored or deleted by the software as they will always differ in the stereo pair. Therefore, the difference in markers will affect the alignment of the two images, causing the resulting three dimensional image to be blurred or distorted, which makes diagnosis more difficult and less certain. The unwanted pixels module 98 deletes these left and right markers.

Next, the transform right image module 100 transforms the image in the right pane, which may be either the left or right digital image, to the three dimensional, that is, XYZ space, of the image in the left pane. This process includes locating keypoints in both the right and left image and using those keypoints to extract descriptors of each image. A keypoint is a pixel that is mathematically significantly different in color or intensity than the twenty-six nearest pixels. The keypoints of each image are used to extract descriptors from each image. Descriptors describe the directional trend of pixels. Descriptors from the image in the left pane are matched with descriptors from the image in the right pane. The descriptors are used to match in the right image to points in the left image, that is, to find points that are in common in the two images, so that the right and left images can be aligned by aligning the points in common in the two images. Then the two images share the same X-axis and Y-axis. The matches in the descriptors are filtered, to remove false positives, resulting in improved alignment of the two images. The descriptors that remain after filtering are used to relocate into the apparent XYZ space of the left image to move the right image into the space of the left image and warp the right image so that it fits into the space of the left image and has the same spatial attributes.

To address privacy and security concerns, the verify digital signature module 102 automatically retrieves the required digital signature files from storage media. If the software is unable to retrieve the required digital signature file, or the file fails verification, then the user 112 is prompted to browse to the location of digital signature files. The verify digital signature module 102 is executed prior to the transform right image module 100, thus alignment and viewing of the source X-ray images is disabled until the required digital signature files are provided.

Immediately after digital signature module 102 verifies valid digital signature files, the interlace images module 106 is invoked and the left digital X-ray image is interlaced with the image from the Transform Right Image module 100. The two images can be interlaced in any known technique. Interlacing is a way to combine two images where one image is displayed on all odd horizontal lines and the other images is displaced on all even horizontal lines of a computer monitor, television or the like. The final display is a computer screen, television or the like whose display can accept and display signals on only certain portions of the screen that can be controlled by electronics. In the present case, the odd numbered rows of the left X-ray image are mixed or interlaced with the even numbered rows of the transformed right digital X-ray image. This results, ultimately, in two separate images that are spaced apart but have certain points in common to insure that the two images are aligned with one another. Alternatively, the left digital X-Ray image could be combined with the right digital X-ray image using active shutter glasses or a head mounted display with separate displays. The two images can be displayed side-by-side, or with one image above the other, in both cases, with a space between them, or as a single stereoscopic three dimensional image.

The align interlaced images module 108 allows the user 112 to align the two images along both the X-axis and the Y-axis, if desired. The align interlaced images module 108 can be useful when the initial alignment cannot be properly performed because the original source images are compromised or the original alignment effort was not entirely successful. This option improves final image quality but is unnecessary in most applications.

In the save interlaced JPG (or JPEG) module 110, the final image is saved as a computer file in any convenient format, which may be in the same file type as photographs, i.e., a JPG file, or JPEG (Joint Photography Experts Group) file or the like. Then this image can be displayed on a television screen, computer monitor 116, or the like as described above at any convenient place. Naturally the resulting files can be sent electronically to anyone desired, but, of course, the user 112 or viewer viewing the files must have a compatible computer display, that is, one that displays interlaced images in the same fashion as the system is designed for and that the user or viewer 112 must wear appropriate three dimensional view eyeglasses 114.

Referring to FIG. 5, the Use Case Diagram shows the user's 112 interaction with the primary modules of the software described above, including the view copyright notices module and attribution for third party components modules 90, the view end user license agreement module 92, the load images module 94, the swap images module 96, the interlace images module 106, the align interlaced images module 108, the save JPG (JPEG)(or other image file module) 11. This diagram illustrates that user involvement is needed to manipulate the images into the desired file that can be viewed as a three-dimensional display on a suitable terminal. The user 112 also interacts with the remove unwanted pixels module 98, as indicated in FIG. 4. The user 112 typically works at a desk 120 with a computer and monitor 116 and keyboard 118, all of which may be wireless, and views the resulting images in three dimensions by wearing the three dimensional viewing eyeglasses 114.

The interlaced stereo three dimensional image can be viewed in three dimensional space in real time in the alignment viewer 112, that is, the computer monitor 116 that the user is viewing, or at any time by viewing the saved JPEG file of the image. A passive three dimensional display and passive three dimensional viewing glasses are required for viewing the final JPG (JPEG) image as a three dimensional image. More than one user 112 can view the three dimensional images at one time so long as each user 112 is wearing the appropriate three dimensional viewing eyeglasses 114, which enhances collaboration and obtaining second opinions in making a diagnosis in a particular case.

While the present invention has been described in accordance with the preferred embodiments thereof, the description is for illustration only and should not be construed as limiting the scope of the invention. Various changes and modifications may be made by those skilled in the art without departing from the spirit and scope of the invention as defined by the following claims.

We claim:

1. A process for creating a three dimensional X-ray image of a subject comprising the steps of:
a. restraining a subject in a desired position;
b. positioning an X-ray emitter in a first position such that the X-ray beam passes through the subject and into an X-ray detector, creating a first X-ray image on said X-ray plate;
c. positioning said X-ray emitter in a second position such that the X-ray beam passes through the subject and into said X-ray detector, creating a second X-ray image on said X-ray plate;
d. manipulating said first X-ray image to align said first X-ray image with said second X-ray image to product an image that can be viewed by a person as a three-dimensional image.

2. A process for creating a three dimensional X-ray image of a subject in accordance with claim 1 wherein the step a of restraining the subject further comprises the steps of:
a. positioning a subject in a posture that permits an X-ray of the desired portion of the subject in the desired orientation;
b. pivoting a right restraint device arm into a position where at least one strap attached to said right restraint device arm can cross a prominent portion of the subject;
c. pivoting a left restraint device arm into a position where said at least one strap will receive said at least one strap such that the subject will be restrained if said strap is tight; and
d. tightening said at least one strap and fixing said at least one strap to said left restraint device arm.

3. A process for creating a three-dimensional X-ray image of a subject in accordance with claim 2 wherein step a of restraining the subject further comprises the steps of:
a. adjusting the position of a left sliding adjustment collar on said left arm of said restraint device; and
b. adjusting the position of a right sliding adjustment collar on said right arm of said restraint devices such that said restraint strap will can be tightened to restrain a subject in a desired position, with said steps a and b being performed prior to step d in claim 2.

4. A process for creating a three dimensional X-ray of a subject in accordance with claim 2 further comprising the step of pressing said subject against an X-ray detector prior to tightening said strap.

5. A process for creating a three-dimensional X-ray image of a subject in accordance with claim 1 wherein wherein said X-ray detector is a digital X-ray detector.

6. A process for creating a three-dimensional X-ray image of a subject in accordance with claim 5 further comprises saving said first X-ray image as a digital file on a computer and saving said second X-ray image as a separate digital file on said computer.

7. A process for creating a three-dimensional X-ray image of a subject in accordance with claim 6 further comprises the steps of aligning said first X-ray image and said second X-ray image along their X and Y axes to create an image that can be viewed as a three dimensional image by a user wearing three dimensional viewing eyeglasses.

8. A process for creating a three dimensional X-ray image of a subject in accordance with claim 1 wherein steps c further comprises taking one X-ray image with said X-ray emitter in a first position, saving the resulting digital file, then moving said X-ray emitter to a second position and taking another X-ray image and saving the resulting digital file.

9. A process for creating a three dimensional X-ray image of a subject in accordance with claim 8 further comprising marking said first X-ray image with a marker indicating left or right and marking said second X-ray image with a marker indicating left or right, with one unique identifier assigned to each said X-ray image.

10. A process for creating a three dimensional X-ray image of a subject in accordance with claim 1 wherein step d further comprises the steps of:
a. aligning said first X-ray image and second X-ray image along their X-axes;
b. interlacing said first and second X-ray images after they have been aligned.

11. A process for creating a three dimensional X-ray image of a subject in accordance with claim 10 further comprising the step of aligning said first X-ray image and said second X-ray image along their Y-axes after step a and prior to step b.

12. A process for creating a three dimensional X-ray image of a subject in accordance with claim 11 further comprising the step of aligning said interlaced images.

13. A process for creating a three dimensional X-ray image of a subject in accordance with claim 10 further comprising the step of removing said unique markers for left and right in said first X-ray image and in said second X-ray image prior to step a.

14. A process for creating a three dimensional X-ray image of a subject in accordance with claim 13 further comprising the step of transforming said first X-ray image or said second X-ray image into the space of the other X-ray image so that the two images may be aligned.

15. A process for creating a three dimensional X-ray image of a subject comprising the steps of:
a. positioning a subject in a posture that permits an X-ray of the desired portion of the subject in the desired orientation;
b. pivoting a right restraint device arm into a position where at least one strap attached to said right restraint device arm can cross a prominent portion of the subject;
c. pivoting a left restraint device arm into a position where said at least one strap will receive said at least one strap such that the subject will be restrained if said strap is tight;
d. tightening said at least one strap and fixing said at least one strap to said left restraint device arm;
e. positioning an X-ray emitter in a first position such that the X-ray beam passes through the subject and into a digital X-ray detector, actuating said X-ray emitter, thereby creating a first X-ray image on said digital X-ray detector;
f. positioning said X-ray emitter in a second position such that the X-ray beam passes through the subject and into said digital X-ray detector, actuating said X-ray emitter, creating a second X-ray image on said digital X-ray detector;
g. aligning said first X-ray image and said second X-ray image;
h. interlacing said first X-ray image and said second X-ray image to create an image that can be displayed on a computer monitor and viewed by a person as a three-dimensional image when the person is wearing three dimensional viewing eyeglasses.

16. A process for creating a three dimensional X-ray image of a subject in accordance with claim 15 further comprising the step of removing unwanted pixels from said first X-ray image and from said second X-ray image prior to the step of aligning said first X-ray image and said second X-ray image.

17. A process for creating a three dimensional X-ray image of a subject in accordance with claim 15 wherein step g further comprises aligning said first X-ray image and said second X-ray image along the X-axes of said first X-ray image and the X-axis of said second X-ray image.

18. A process for creating a three dimensional X-ray image of a subject comprising the steps of:
a. positioning a subject in a posture that permits an X-ray of the desired portion of the subject in the desired orientation;
b. pivoting a right restraint device arm into a position where at least one strap attached to said right restraint device arm can cross a prominent portion of the subject;
c. pivoting a left restraint device arm into a position where said at least one strap will receive said at least one strap such that the subject will be restrained if said strap is tight;
d. tightening said at least one strap and fixing said at least one strap to said left restraint device arm;
e. positioning an X-ray emitter in a first position such that the X-ray beam passes through the subject and into a digital X-ray detector, actuating said X-ray emitter, thereby creating a first X-ray image on said digital X-ray detector;
f. positioning said X-ray emitter in a second position such that the X-ray beam passes through the subject and into said digital X-ray detector, actuating said X-ray emitter, creating a second X-ray image on said digital X-ray detector;
g. aligning said first X-ray image and said second X-ray image along an X-axis of each image;
h. aligning said first X-ray image aligning said first X-ray image and said second X-ray image along the Y-axes of said first X-ray image and said second X-ray image;
i. interlacing said first X-ray image and said second X-ray image to create an image that can be displayed on a computer monitor and viewed by a person as a three-dimensional image when the person is wearing three dimensional viewing eyeglasses.

19. A process for creating a three dimensional X-ray image of a subject in accordance with claim 18 further comprising the step of removing unwanted pixels from said first X-ray image and from said second X-ray image prior to the step g of aligning said first X-ray image and said second X-ray image.

20. A process for creating a three dimensional X-ray image of a subject in accordance with claim 18 further comprising the steps of aligning said interlaced X-ray images after the step of interlacing said fir X-ray image and said second X-ray image.
